(19) United States
(12) Patent Application Publication
(10) Pub. No.: US 2026/0187730 A1
(43) Pub. Date: Jul. 2, 2026

(54) System and Method for Processing an Explanation of Benefits Statement from an Insurance Carrier to a Healthcare Provider

(71) Applicants: Joon Kim, Germantown, MD (US); Hyesun Jee, Germantown, MD (US)

(72) Inventors: Joon Kim, Germantown, MD (US); Hyesun Jee, Germantown, MD (US)

(21) Appl. No.: 19/433,566

(22) Filed: Dec. 26, 2025

Related U.S. Application Data
(60) Provisional application No. 63/739,475, filed on Dec. 27, 2024.

(51) Int. Cl. G06Q 40/08; G16H 10/60

(52) U.S. Cl. CPC G06Q 40/082; G16H 10/60

(57) ABSTRACT

A system and method for processing an Explanation of Benefits (EOB) statement from an insurance carrier to a healthcare provider facilitates the processing of an EOB statement. The system sorts out and inputs the correct Coordination of Benefits (COB) to make billing payment entry mostly automatic, only requiring user input when necessary. The EOB statement is imported and sliced into a patient-specific statement. Desired data elements are extracted from the patient-specific statement using optical character recognition (OCR) to generate a machine-readable statement. The patient-specific statement is normalized into a standardized patient-specific statement (SPSS), and COB is performed based on the SPSS. A summary report is generated based on the COB and the SPSS. Further, the patient-specific statement, the SPSS, and the summary report are relayed to a remote server and a healthcare provider server. The patient-specific statement, the SPSS, and the summary report are inserted into the corresponding patient account.

### FIELD OF THE INVENTION

The present invention relates generally to dental billing systems and Revenue Cycle Management (RCM) systems. More specifically, the present invention provides means for inputting data of an Explanation of Benefit (EOB) statement from insurance carriers to healthcare provider software. The present invention calculates, validates, and alerts the appropriate users of errors.

### BACKGROUND OF THE INVENTION

An Explanation of Benefit (EOB) statement is a document that insurance companies sends after processing a medical or dental claim. The EOB statement is sent to the corresponding patient and healthcare provider. An Electronic Remittance Advice (ERA) file is an EOB statement in electronic format. When a healthcare provider receives an EOB statement, the EOB statement usually includes multiple pages of claim-related information. Generally, the first page of the EOB statement includes explanations about the performed procedures with the relevant billing amounts calculated. Another page of the EOB statement has total amount paid and code and the corresponding remarks that explain why some procedures were not paid or denied. These codes and remarks are often the only means for insurance carriers to communicate with the healthcare provider regarding the carriers' decisions. These codes are computer generated, and remarks are often not very comprehensive. Lastly, the EOB statement includes a check payment. If an ERA file is provided, the payment would usually be performed via direct deposit into the corresponding bank account via Electronic Funds Transfer (EFT).

Moreover, EOB statements often include information for multiple patients primarily due to insurance carriers trying to save postage and paper. This further complicates the processing of the EOB statement by the healthcare provider, which may simultaneously receive several EOB statements in a short period of time. Therefore, a computing system that facilitates the processing of the EOB statement by calculating the corresponding payment values, verifying the calculated payment values, and alerting the appropriate users of any potential errors is beneficial and necessary to the healthcare provider.

### SUMMARY OF THE INVENTION

The present invention provides a system and method for processing an Explanation of Benefits (EOB) statement from an insurance carrier to a healthcare provider. The system of the present invention is designed to be able to process a physical EOB statement received via mailed or faxed, or Electronic Remittance Advice (ERA) obtained from the carrier's website. The EOB statement is processed to generate digital file that can stored on a network computing device that is equipped with the software system of the present invention. The software system of the present invention is designed to enable the processing of the EOB statement according to the method of the present invention. The overall process of the method of the present invention is generally performed automatically with the appropriate prompts to the appropriate users whenever user input is necessary.

In the preferred embodiment, the overall process of the method of the present invention involves converting the digital EOB statement to a machine-readable file using Optical Character Recognition (OCR). Specific data points are identified and normalized into a standardized format. This is an important step to facilitate the communication of the software system with different healthcare providers. In addition, EOB statements with several patient claims are separated into several patient-specific statements, each including the corresponding patient's information from the original EOB statement. Separating the EOB statement into patient-specific statements allows the system to avoid Health Insurance Portability and Accountability Act (HIPPA) violations, facilitates organizing patient records, and makes retrieving and relaying patient-specific information a simple process.

Moreover, the overall process of the method of the present invention facilitates the process of Coordination of Benefits (COB) for each patient claim provided on the EOB statement. The system allows the sorting of primary and secondary insurances for each patient claim if the corresponding patient has multiple insurance plans. In addition, the system is capable of interpretating, calculating, inputting the appropriate data, and ultimately alerting the appropriate users of errors in the corresponding claim and suggesting potential solutions (manual input or suggested input). Additional features and benefits of the system and method of the present invention are further discussed in the sections below.

### BRIEF DESCRIPTION OF THE DRAWINGS

FIG. 1 is a box diagram showing the overall system of the present invention.

FIG. 2 is a flowchart illustrating the overall method of the present invention.

FIG. 3 is a flowchart illustrating the continuation of the overall method of the present invention shown in FIG. 2.

FIG. 4 is a flowchart illustrating the subprocess of processing a physical Explanation of Benefits (EOB) statement.

FIG. 5 is a flowchart illustrating the subprocess of processing a digital EOB statement.

FIG. 6 is a flowchart illustrating the subprocess of generating several patient-specific statements from an EOB statement.

FIG. 7 is a flowchart illustrating the subprocess of de-identifying patient information for OCR processing.

FIG. 8 is a flowchart illustrating the subprocess of normalizing the desired claim data points.

FIG. 9 is a flowchart illustrating the subprocess of verifying the identified claim data points.

FIG. 10 is a flowchart illustrating the subprocess of performing COB for the patient-specific statement if the corresponding patient has a single insurance plan.

FIG. 11 is a flowchart illustrating the subprocess of calculating primary insurance values for a patient-specific statement with a corresponding primary insurance plan.

FIG. 12 is a flowchart illustrating the subprocess of determining if the patient-specific statement corresponds to a primary medical plan or a primary dental plan.

FIG. 13 is a flowchart illustrating the subprocess of performing COB for a patient-specific statement with a primary medical insurance plan.

FIG. 14 is a flowchart illustrating the subprocess of performing COB for a patient-specific statement with a primary dental insurance plan and the healthcare provider being in-network.

FIG. 15 is a flowchart illustrating the subprocess of performing COB for a patient-specific statement with a primary dental insurance plan and the healthcare provider being out-of-network.

FIG. 16 is a flowchart illustrating the subprocess of determining if the patient-specific statement corresponds to a secondary EOB statement and determining if a corresponding primary EOB statement has been processed.

FIG. 17 is a flowchart illustrating the subprocess of performing COB for a patient-specific statement with a corresponding primary medical insurance plan and a secondary dental insurance plan.

FIG. 18 is a flowchart illustrating the subprocess of performing COB for a patient-specific statement with a corresponding primary dental insurance plan and an out-of-network secondary dental insurance plan.

FIG. 19 is a flowchart illustrating the subprocess of performing COB for a patient-specific statement with a corresponding primary dental insurance plan and an in-network secondary dental insurance plan.

FIG. 20 is a flowchart illustrating the subprocess of including reimbursement values in the summary report.

FIG. 21 is a table showing the standardized data points for claim data points of exemplary insurance carriers.

### DETAIL DESCRIPTIONS OF THE INVENTION

All illustrations of the drawings are for the purpose of describing selected versions of the present invention and are not intended to limit the scope of the present invention.

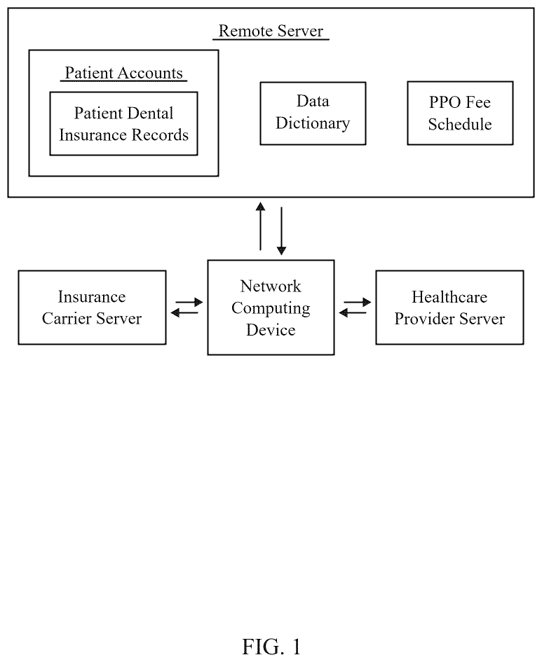

The present invention provides a system and method for processing an Explanation of Benefits (EOB) statement from an insurance carrier to a healthcare provider. The present invention facilitates the processing of an EOB statement by sorting out and inputting the correct Coordination of Benefits (COB) to make billing payment entry mostly automatic, only requiring user input whenever necessary. In the preferred embodiment, the system of the present invention includes at least one network computing device (NCD) managed by a remote server (Step A), as can be seen in FIG. 1. The NCD is any computing device that can connect to the Internet that allows an authorized user to monitor and control the processing of the EOB statement. For example, the NCD can include, but is not limited to, desktop computers, laptop computers, tablet computers, smartphones, etc.

The remote server is a server independent from the healthcare provider and the insurance carrier. The remote server also includes a plurality of patient accounts that facilitate the organization and processing of the patient claims. The remote server also stores processed claims to properly record all data for auditing, such as records audits or compliance audits. Further, the NCD is connected to at least one Healthcare Provider Server (HPS) that corresponds to the server that facilitates the connection of the system of the present invention to the healthcare provider system. In addition, the system includes at least one EOB statement (Step B), as can be seen in FIG. 1. The EOB statement corresponds to the statement being processed by the system of the present invention. The EOB statement includes dental billing information for at least one patient account corresponding to claim information for a patient of the healthcare provider. Moreover, the EOB statement can be provided in different formats that can be accessed and imported into the NCD for processing. Depending on the format the EOB statement is provided, different methods can be used to convert the EOB statement into a corresponding format that can be utilized by the system

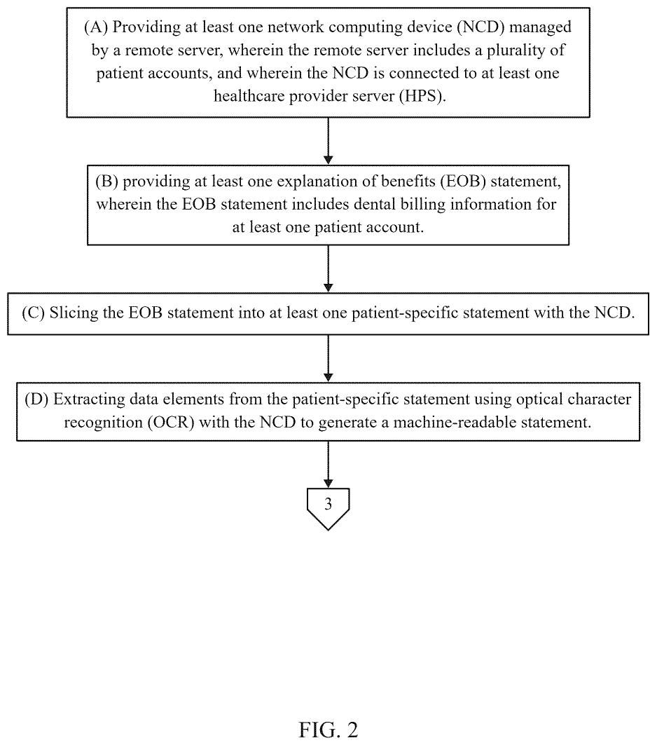

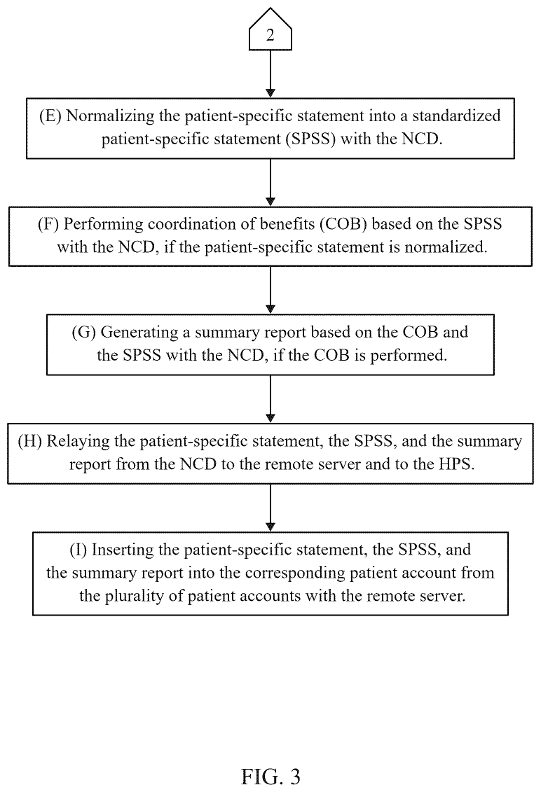

The system of the present invention enables the performance of an overall process of the method of the present invention. As can be seen in FIGS. 2 and 3, the overall process of the method of the present invention begins by slicing the EOB statement into at least one patient-specific statement with the NCD (Step C). The patient-specific statement includes only the claim information for a specific patient of the healthcare facility to meet specific privacy protocols. The patient-specific statement is stored locally for processing and can be relayed to the HPS for the provider's records. Then, the desired data elements are extracted from the patient-specific statement using Optical Character Recognition (OCR) with the NCD to generate a machine-readable statement (Step D). Depending on the necessary calculations the system must perform, the data elements extracted can vary. Once the machine-readable version of the patient-specific statement is generated, the patient-specific statement is normalized into a standardized patient-specific statement (SPSS) with the NCD (Step E). The SPSS is normalized into a standardized format that facilitates the communication of the system of the present invention with the different healthcare provider systems. Then, COB is performed based on the SPSS with the NCD (Step F), if the patient-specific statement is normalized.

Different proprietary calculations are performed by the system during the COB to sort out primary and secondary insurances, interpret procedure codes, calculate insurance payment values, validate the calculated values, alert the user of errors in the calculations, and relaying the appropriate data into the corresponding healthcare provider system. In addition, a summary report is generated based on the COB results and the SPSS with the NCD (Step G), if the COB is performed, as can be seen in FIGS. 2 and 3. The summary report is a summarization of the patient-specific statement and the COB results that informs authorized users of the billing process results. Then, the patient-specific statement, the SPSS, and the summary report are relayed from the NCD to the remote server and to the HPS (Step H). This way, authorized users such as the healthcare provider or billing administrator can follow up on the daily transactions. Moreover, the patient-specific statement, the SPSS, and the summary report are inserted into the corresponding patient account from the plurality of patient accounts with the remote server (Step I). This step is particularly important for accountability and financial transparency.

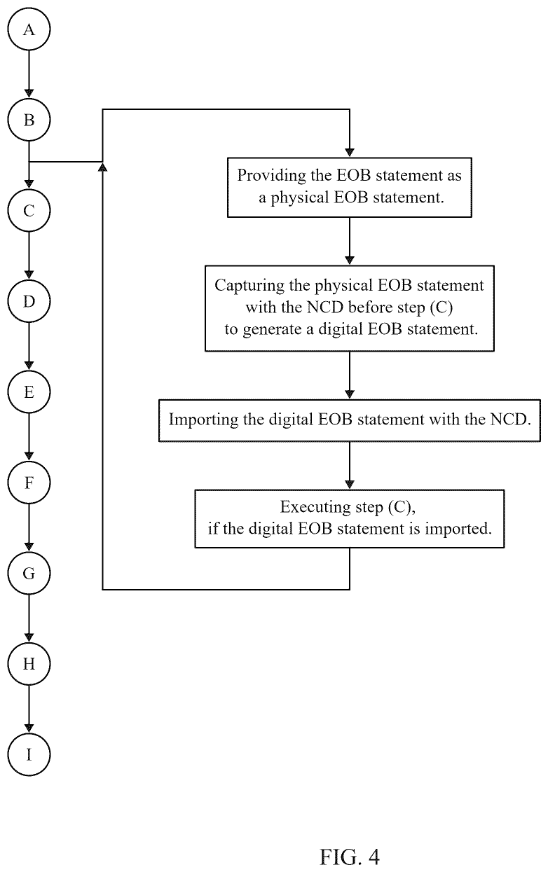

As previously discussed, the system of the present invention can process the EOB statement even when provided in different formats by the insurance carrier. In some embodiments, the EOB statement is provided as a physical EOB statement the insurance carrier sends to the healthcare provider via mail, fax, etc. As can be seen in FIG. 4, the subprocess of processing a physical EOB statement includes the steps of capturing the physical EOB statement with the NCD before Step C to generate a digital EOB statement. The capture of the physical EOB statement can be performed in different ways depending on the capabilities of the NCD. For example, for desktop computers or laptops, a scanner can be utilized to scan the physical EOB statement. If using a mobile device, the physical EOB statement can be scanned using the integrated camera. Once the digital EOB statement is generated, the digital EOB statement is imported with the NCD to perform the overall process. Then, Step C is executed, if the digital EOB statement is imported.

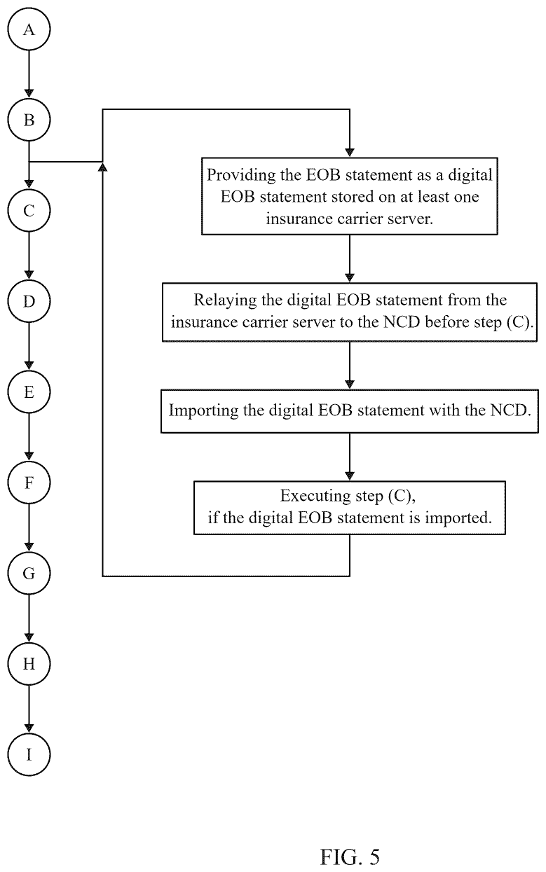

In other embodiments, the EOB statement is provided as a digital EOB statement provided by the insurance carrier. For example, the digital EOB statement can be downloaded from the carrier's system, received via email, or downloaded using secure servers. The insurance carrier can include at least one insurance carrier server from which the digital EOB statement can be retrieved from. As can be seen in FIG. 5, the subprocess of processing a digital EOB statement includes the steps of relaying the digital EOB statement from the insurance carrier server to the NCD before Step C. For example, the digital EOB statement can be provided via Electronic Remittance Advice (ERA) from the carrier. The digital EOB statement is imported with the NCD for use for the overall process. Then, Step C is executed, if the digital EOB statement is imported so that the system can carry out the overall process.

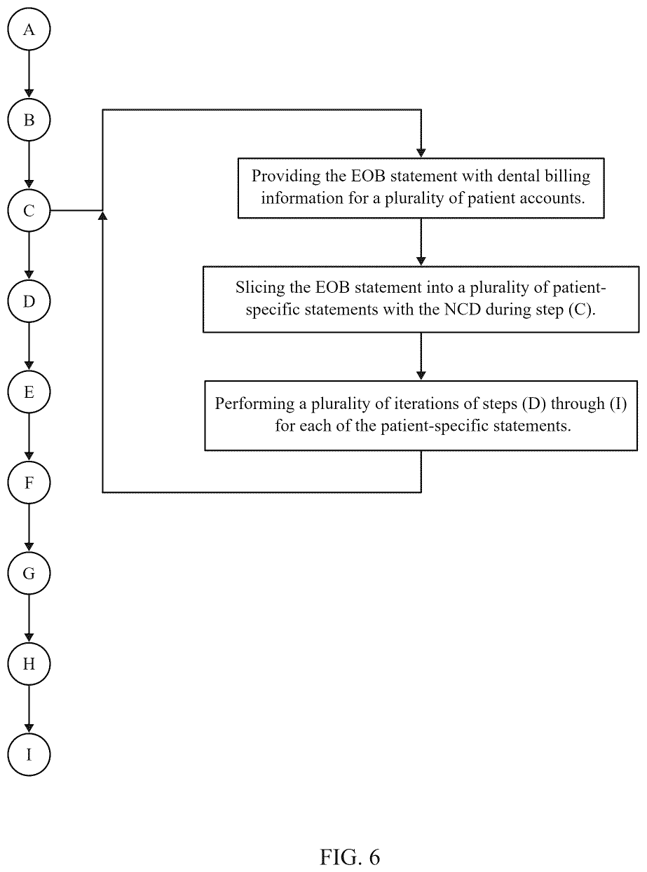

Regardless of the format the digital EOB statement being imported with the NCD, the system of the present invention can convert the EOB statement into a machine-readable statement to access the necessary information to perform the necessary calculations during the COB process. In addition, as mentioned before, the EOB statement can include dental billing information for a plurality of patient accounts. Insurance carriers often provide billing information for multiple patients to reduce administration costs. This becomes a HIPPA violation when the end user, particularly the healthcare provider's administrator, stores the EOB statement into the healthcare provider software or sends out secondary insurance claims. As can be seen in FIG. 6, the subprocess of generating several patient-specific statements from an EOB statement includes the steps of slicing the EOB statement into a plurality of patient-specific statements with the NCD during Step C. The table header of the EOB statement is segmented and duplicated into each corresponding patient-specific entry. Then, the resulting patient-specific entries are merged into a consolidated patient-specific statement. Once the patient-specific statements are generated, a plurality of iterations of Steps D through I are performed for each of the patient-specific statements. In other words, each patient-specific statement is processed, and the COB process is performed for each patient claim.

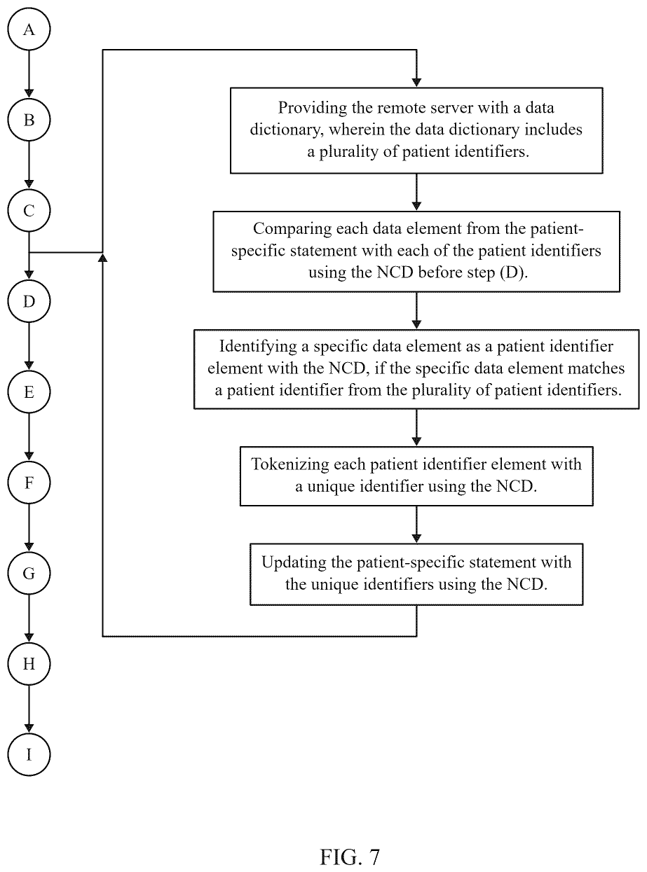

Different OCR algorithms, either proprietary or from third-party services, can be utilized to generate the machine-readable statement. To further follow HIPPA regulations, all patient identifiers are removed and substituted with universally unique identifiers and hashed tokens to maintain HIPAA-compliant de-identification when using machine learning or Artificial Intelligence (AI) for OCR processing. To do so, the remote server is provided with a data dictionary that includes a plurality of patient identifiers which can be used to anonymize patient information. As can be seen in FIG. 7, the subprocess of de-identifying patient information for OCR processing includes the steps of comparing each data element from the patient-specific statement with each of the patient identifiers using the NCD before Step D. The patient identifiers can include, but are not limited to, patient name, provider's address, billing information, account number, etc. Then, a specific data element is identified as a patient identifier element with the NCD, if the specific data element matches a patient identifier from the plurality of patient identifiers. Once the patient identifier elements are identified, each patient identifier element is tokenized with a unique identifier using the NCD to de-identify all patient identifiers. Finally, the patient-specific statement is updated with the unique identifiers using the NCD which can then be sent for OCR. In other embodiments, other privacy processes can be performed to comply with HIPPA regulations.

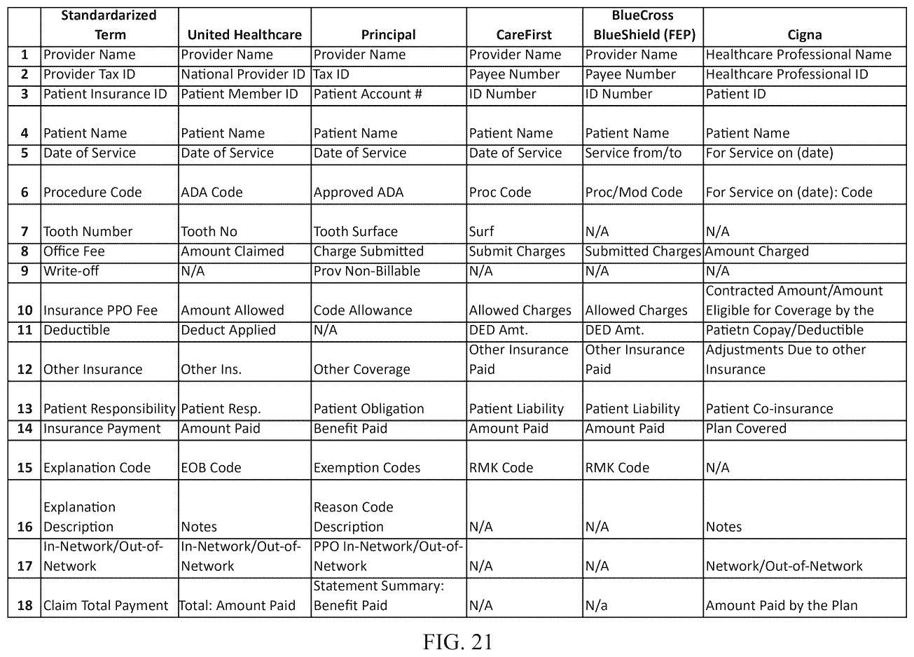

As previously discussed, specific claim data points are extracted from the patient-specific statement to perform the COB processes. To do so, the data dictionary further includes a plurality of claim data points and a plurality of standardized data points. The claim data points correspond to the specific claim data points the system needs to process the patient-specific statement. As can be seen in FIG. 21, the standardized data points correspond to the normalized claim data points that are used to the facilitate the communication between the system and different healthcare provider systems. In addition, each of the claim data points is associated with a corresponding standardized data point that allows the system to map the claim data points to the equivalent standardized data point.

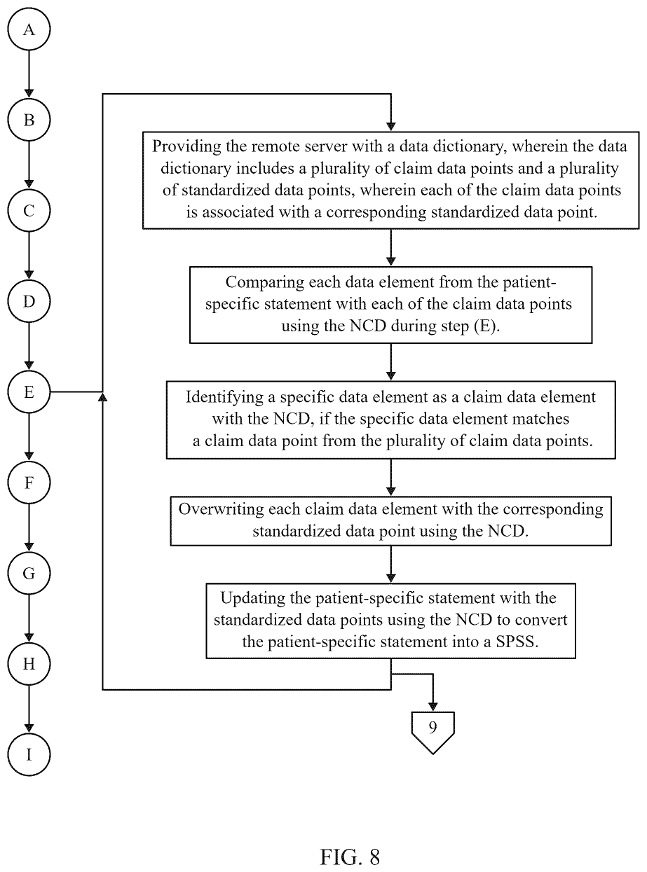

As can be seen in FIG. 8, the subprocess of normalizing the desired claim data points includes the steps of comparing each data element from the patient-specific statement with each of the claim data points using the NCD during Step E. Then, a specific data element is identified as a claim data element with the NCD, if the specific data element matches a claim data point from the plurality of claim data points. The number of claim data points can vary depending on the specific values necessary to perform the COB processes. Once the claim data elements are identified, each claim data element is overwritten with the corresponding standardized data point using the NCD. These standardized data points are shared with healthcare providers to help the authorized users understand the claim information provided by the insurance carrier. Then, the patient-specific statement is updated with the standardized data points using the NCD to convert the patient-specific statement into a SPSS.

In the preferred embodiment, the claim data points includes 18 non-standardized data points which the system converts into standardized data points, as can be seen in FIG. 21. The standardized data points include the provider's name, provider tax Identification (ID), patient insurance ID, patient name, date of service, procedure code, tooth number, office fee, write-off, insurance Preferred Provider Organization (PPO) fee, deductible, other insurance, patient responsibility, insurance payment, explanation code, explanation description, In-Network (IN)/Out-of-Network (OON), and claim total payment. Normalizing non-standardized data points can be difficult due to carriers using completely different names for the same data points. Healthcare providers regularly deal with numerous healthcare plans from various insurance carriers. However, healthcare providers only have the telephone, fax, or letters available as a mean of communicating with carriers. Therefore, normalizing non-standardized data points and other patient information is necessary.

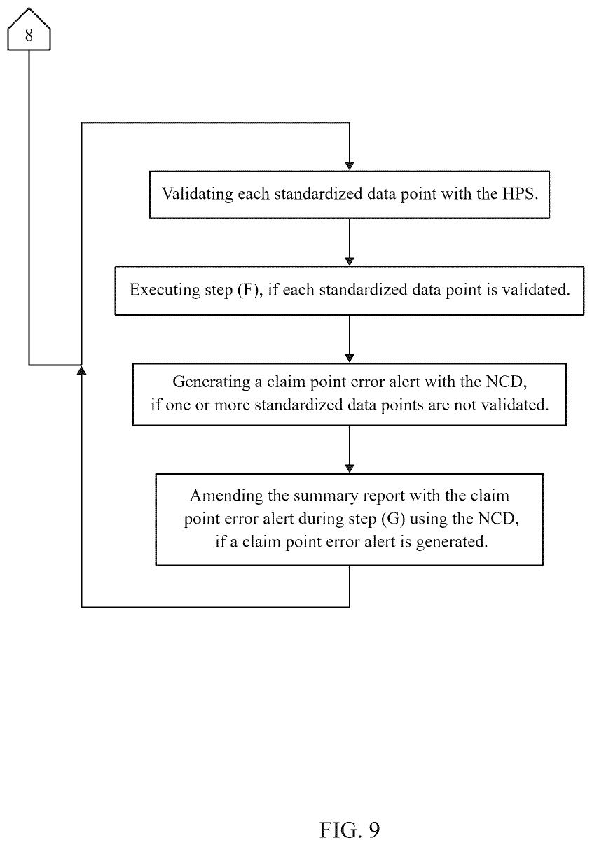

After identifying and normalizing the desired claim data points, the system of the present invention validates the corresponding values to ensure that the provided values match with the healthcare provider's records. This is necessary to ensure that the carrier's claim payment matches the provider's claim values. As can be seen in FIG. 9, the subprocess of verifying the identified claim data points includes the steps of validating each standardized data point with the HPS. The provider's records are used to validate the values provided on the SPSS to prevent calculation errors during the COB processes. Then, Step F is executed if each standardized data point is validated. By validating all necessary claim data points, calculation errors are prevented during the COB processes. Otherwise, a claim point error alert is generated with the NCD, if one or more standardized data points are not validated. The claim point error alert is outputted by the NCD to alert the corresponding user of the claim point error. The corresponding user can also be prompted to input or correct the corresponding claim data points. In addition, the summary report is amended with the claim point error alert during Step G using the NCD, if a claim point error alert is generated. This saves the claim point error alert for future auditing. In other embodiments, different claim data point verification methods can be implemented as necessary.

As previously mentioned, the system of the present invention can sort through primary and secondary insurances during the COB processes. Many patients are insured through multiple insurers. For example, federal employees have medical insurance that partially covers dental benefits, and others have additional insurance coverage when claimed by family members. In general, when there is more than one insurance involved, first insurance that claims go through is considered primary insurance and proceeding insurance becomes a secondary insurance. For example, for federal employees medical becomes primary insurance and dental insurance becomes secondary insurance. Federal employees are the only patients the system needs to consider with medical claims on dental coverage. Other patients' medical insurances do not have any coverage on dental claims. However, Federal government is the largest employer in the United States and consist of about 0.6% of U.S. Population. Moreover, some patients have personal dental insurance and additional insurance from family members. In these situations, the personal dental insurance becomes the primary insurance and family insurance becomes the secondary insurance. Most insurance carriers know if the patient has additional insurance coverage. Therefore, carriers can deny payment or partially pay on the carrier's estimate of what primary insurance pays.

General rule dictates that if there is primary and secondary insurances, healthcare providers are allowed to accept from the insurance carriers toward the provider's fee. However, if the healthcare provider is in-network within the patient insurance plan, providers are contracted to the negotiated fee. Majority of times, primary insurance does not cover the provider's procedure fee and therefore, the secondary insurance covers the remaining portion of the balance. Primary and secondary insurance carriers have previously negotiated fees and coverage. When secondary insurance makes payments, the EOB statement is written as if the EOB statement is a stand-alone insurance like primary insurance, but the COB payment is calculated on the patient's secondary plan. There is a discrepancy on the EOB statement line by line payments and total actual payment on the claim. Therefore, the data from the EOB statement cannot be input into the healthcare provider software by simply inputting the corresponding alphanumeric values. The system of the present invention sorts this out and inputs the correct COB values and guides the end users along the complicated calculations. Thus, the billing payment entry becomes a generally automatic process, giving end user control only at different steps throughout the process when necessary.

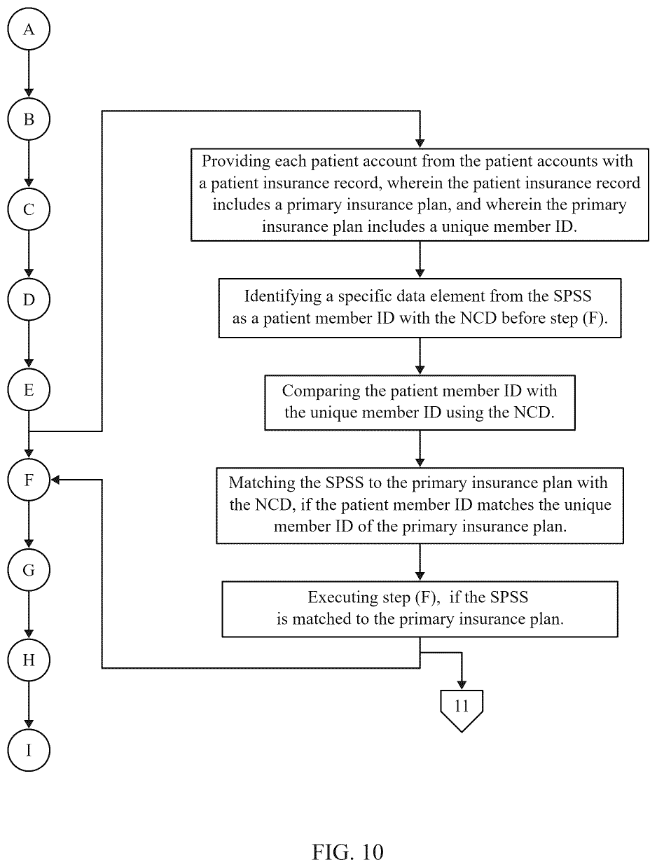

Before performing the COB processes, the system of the present invention determines what insurance coverage the corresponding patient has. To do so, each patient account from the plurality of patient accounts is provided with a patient insurance record. If the patient has a single insurance plan, the patient insurance record includes a primary insurance plan. Further, the primary insurance plan generally includes a unique member Identification (ID) which can be utilized to determine the type of insurance the corresponding patient has. As can be seen in FIG. 10, the subprocess of performing COB for the patient-specific statement if the corresponding patient has a single insurance plan includes the steps of identifying a specific data element from the SPSS as a patient member ID with the NCD before Step F. The identified patient member ID is then compared with the unique member ID using the NCD. This allows the system to confirm the insurance the corresponding patient has. Further, the SPSS is matched to the primary insurance plan with the NCD, if the patient member ID matches the unique member ID of the primary insurance plan. Then, Step F is executed, if the SPSS is matched to the primary insurance plan by the system.

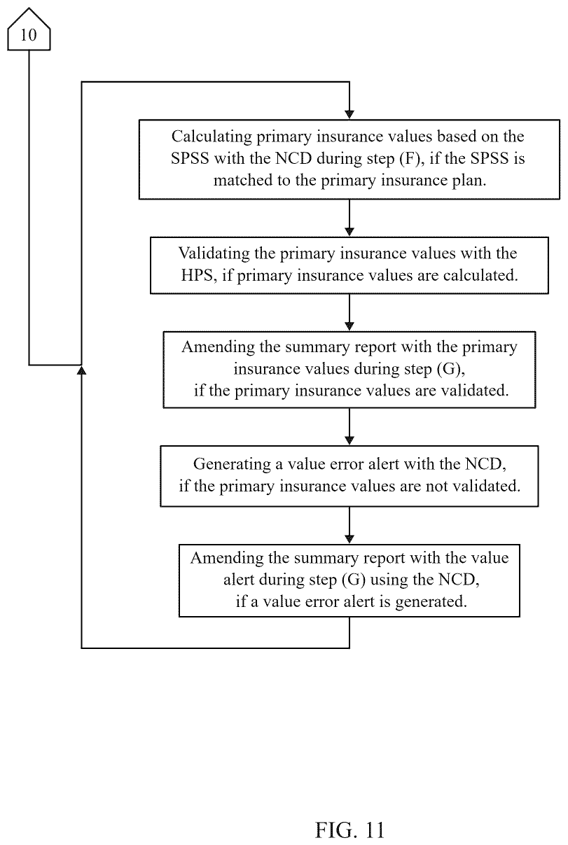

As can be seen in FIG. 11, the subprocess of calculating primary insurance values for a patient-specific statement with a corresponding primary insurance plan includes the steps of calculating the primary insurance values based on the SPSS with the NCD during Step F, if the SPSS is matched to the primary insurance plan. Proprietary calculations are performed using specific predetermined rules. Then, the primary insurance values are validated with the HPS, if primary insurance values are calculated. The primary insurance values are compared with estimated values provided by the healthcare provider system to help validate the accuracy of the primary insurance values. If the primary insurance values are validated, the primary insurance values are inputted into the healthcare provider system, and the summary report is amended with the primary insurance values during Step G. Otherwise, if the primary insurance values are not validated, a value error alert is generated with the NCD.

Like before, the value error alert is output with the NCD to notify the corresponding user. In some embodiments, the corresponding user can be linked to patient account modules on the NCD for the user to review. The corresponding user can also be prompted to manually input the correct primary insurance values using the NCD. Further, the summary report is amended with the value alert during Step G using the NCD, if a value error alert is generated. In the preferred embodiment, the value error alert can be output as a popup that displays various information including, but not limited to, patient name, date of service, tooth number, procedure code, procedure code description (from American Dental Association [ADA] code number description), explanation code, explanation description, as well as a “Go to Account” function that directs the corresponding user to the patient chart on the HPS via an Application Programming Interface (API) connection.

Moreover, the primary insurance values are calculated using a primary insurance calculator which includes the following formulas:

$$OF - PPO fee = WO$$

$$( PPO fee - Deductable ) \times CP = IP$$

$$OF - WO = IP + PR = PPO fee$$

Wherein OF corresponds to office fee, WO corresponds to write-off, CP corresponds to coverage percentage, IP corresponds to insurance payment, and PR corresponds to patient responsibility. In addition, the primary insurance values preferably include, but are not limited to, the office fee, the PPO fee, the write-off, the deductible, the insurance payment, and the patient responsibility. These primary insurance values are compared with the values provided by the healthcare provider system to ensure the accuracy of the values provided by the insurance carrier. Then, the insurance payment, the patient responsibility, and the write-off are input into the healthcare provider system once the calculations are validated. In other embodiments, different insurance values are calculated using different formulas.

As previously discussed, the patients may have multiple insurance coverage that needs to be considered when performing the COB processes. When dealing with patients that have multiple insurance coverage, the patient medical records include a plurality of patient insurance plans, each patient insurance plan including a unique member ID that can be used to identify the patient's corresponding insurance plan. If the patient has two insurance plans, the system of the present invention needs to determine if the SPSS corresponds to a primary insurance or to a secondary insurance. In the preferred embodiment, the patient member ID provided on the SPSS is used to determine which insurance plan the SPSS corresponds to. The HPS can also help verify the patient member ID and determine which insurance plan the SPSS corresponds to. If the patient has two insurance plans and if the matched patient insurance plan is a primary insurance plan, the system needs to also determine if the matched patient insurance plan is a primary medical plan or a primary dental plan.

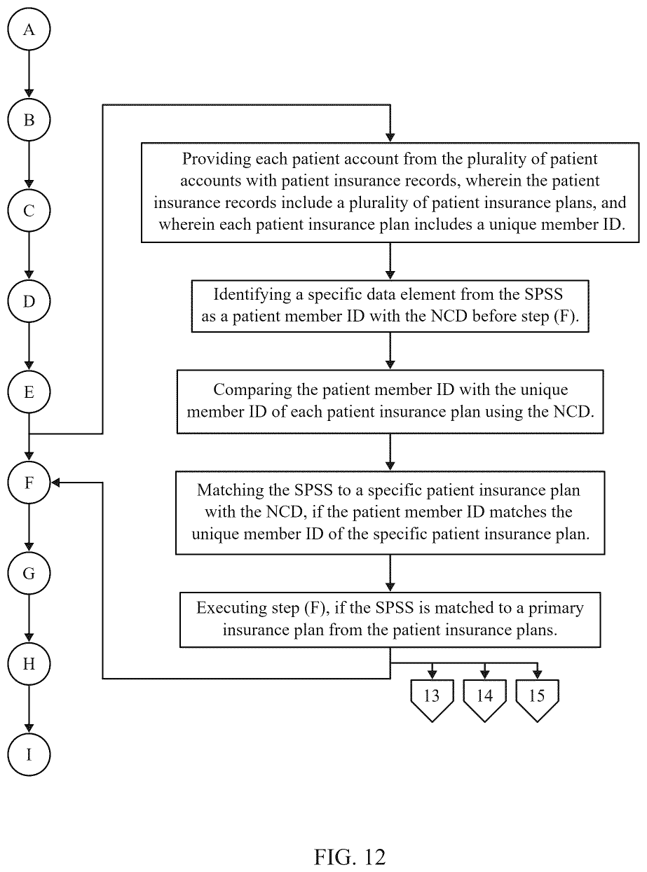

As can be seen in FIG. 12, the subprocess of determining if the patient-specific statement corresponds to a primary medical plan or a primary dental plan includes the steps of identifying a specific data element from the SPSS as a patient member ID with the NCD before Step F. The identified patient member ID is then compared with the unique member ID of each patient insurance plan using the NCD. If the patient member ID matches the unique member ID of the specific patient insurance plan, the SPSS is matched to a specific patient insurance plan with the NCD. Then, Step F is executed, if the SPSS is matched to a primary insurance plan from the patient insurance plans. Depending on the matching results, the corresponding primary insurance plan can be matched to a primary medical insurance plan or a primary dental insurance plan. A medical insurance plan can also be identified using the patient's group number and patient member ID.

When the corresponding primary insurance plan is matched to a primary medical insurance plan, the system needs to determine if the corresponding claim corresponds to procedures that fall under preventive codes or diagnostic codes since medical insurance plans only covers preventive and diagnostic codes for dental procedures. To do so, the data dictionary includes a plurality of valid procedure codes which include a plurality of preventive codes and a plurality of diagnostic codes. The preventive codes and diagnostic codes can include, but are not limited to, comprehensive examination code, periodic examination code, limited examination code, adult prophylaxis code, child prophylaxis code, topical fluoride varnish code, etc.

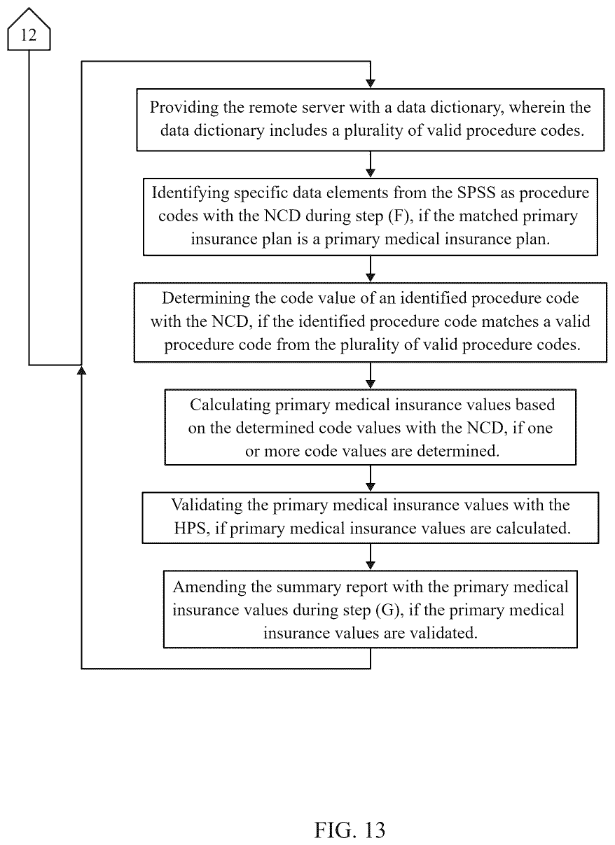

As can be seen in FIG. 13, the subprocess of performing COB for a patient-specific statement with a primary medical insurance plan includes the steps of identifying specific data elements from the SPSS as procedure codes with the NCD during Step F, if the matched primary insurance plan is a primary medical insurance plan. Each identified procedure code is compared with each of the preventive codes and each of the diagnostic codes with the NCD to determine if the identified procedure code matches one of the preventive or diagnostic codes. Then, the code value of an identified procedure code is determined with the NCD, if the identified procedure code matches a valid procedure code from the plurality of valid procedure codes. In other words, the identified procedure code could match a preventive code or a diagnostic code. If one or more code values are determined, the primary medical insurance values are calculated based on the determined code values with the NCD. Like before, the primary medical insurance values are also validated with the HPS, if primary medical insurance values are calculated.

If the primary medical insurance values are validated, the primary medical insurance values are input into the HPS, and the summary report is amended with the primary medical insurance values during Step G, as can be seen in FIG. 13. Alternatively, a medical value error alert is generated with the NCD, if the primary medical insurance values are not validated. The medical value error alert can also be output to the corresponding user with the NCD. Further, the summary report is amended with the medical value error alert during Step G, if a medical value error alert is generated, for future auditing.

In the preferred embodiment, the primary medical insurance values includes the primary insurance payment, the primary patient responsibility, and the office fee. Further, the validation process, the primary medical insurance values are validated using the following formula:

$$OF - PIP - PPR = WO$$

Wherein OF corresponds to office fee, WO corresponds to write-off, PIP corresponds to primary insurance payment, and PPR corresponds to primary patient responsibility. Further, once the primary medical insurance values are inputted into the HPS, the system of the present invention needs to determine if a secondary insurance statement is on hold on the remote server. If there is a secondary insurance statement on hold, the COB process is performed for the secondary dental statement. If there is not a secondary insurance statement on hold, the system needs to determine if the patient's primary insurance plan and secondary insurance plan are from the same insurance carrier. Often, if both primary and secondary insurance plans are from the same insurance carrier, the carrier would take care of the secondary insurance claim statement internally without healthcare provider submitting another claim to the insurance carrier. Regardless, the corresponding user is prompted the option to send a secondary claim to the secondary insurance carrier. If accepted, the corresponding user can be prompted to attach the patient-specific statement to the secondary claim along with other claim files from the HPS. If the second insurance carrier is a different carrier, the corresponding user is also prompted to submit the secondary claim with the patient-specific statement and other claim files.

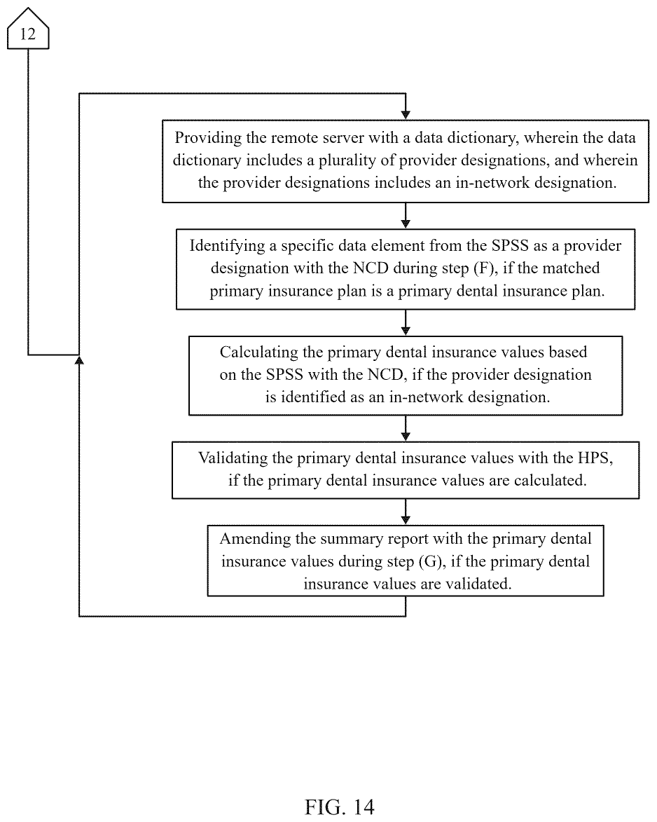

When the corresponding primary insurance plan is matched to a primary medical insurance plan, the system needs to determine if the healthcare provider is in-network or out-of-network with the primary dental insurance plan. To do so, the data dictionary includes a plurality of provider designations that can include an in-network designation and an out-of-network designation. As can be seen in FIG. 14, the subprocess of performing COB for a patient-specific statement with a primary dental insurance plan and the healthcare provider being in-network includes the steps of identifying a specific data element from the SPSS as a provider designation with the NCD during Step F, if the matched primary insurance plan is a primary dental insurance plan. This allows the system to ensure that the corresponding healthcare provider is an in-network provider. If the provider designation is identified as an in-network designation, the primary dental insurance values are calculated based on the SPSS with the NCD. Like before, the primary dental insurance values are validated with the HPS, if the primary dental insurance values are calculated.

In the preferred embodiment, the primary dental insurance values are calculated using the same primary insurance calculator utilized for the primary insurance values. The primary dental insurance values can include, but are not limited to, primary insurance payment, primary write-off, and patient responsibility. Then, the summary report is amended with the primary dental insurance values during Step G, if the primary dental insurance values are validated. In addition, the primary dental insurance values are input into the HPS. Alternatively, a dental value error alert is generated with the NCD, if the primary dental insurance values are not validated. The corresponding user can also be prompted to enter the primary dental insurance values manually. Then, the summary report is amended with the dental value error alert during Step G, if a dental value error alert is generated. Further, the primary dental insurance values are submitted to the corresponding secondary dental insurance plan. The patient-specific statement along with other claim files are attached to the secondary claim.

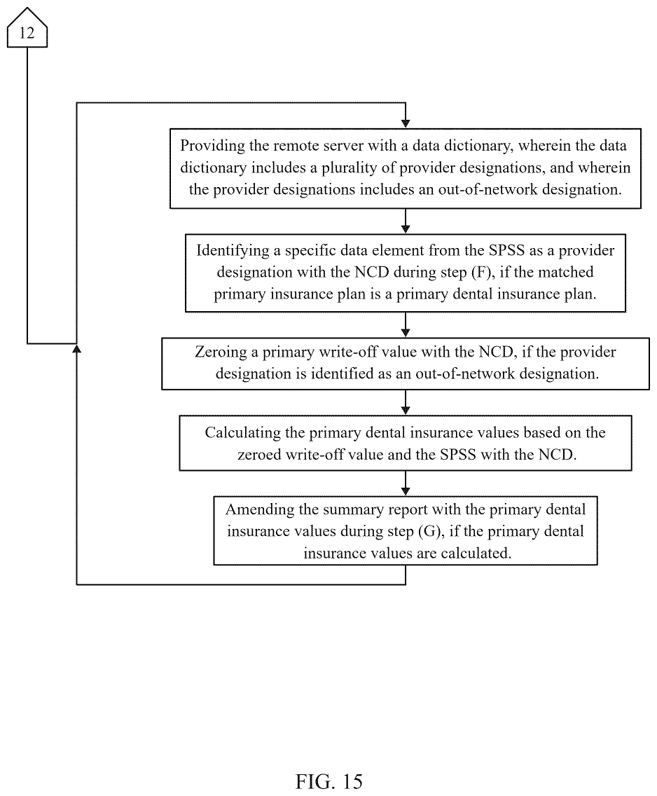

As can be seen in FIG. 15, on the other hand, the subprocess of performing COB for a patient-specific statement with a primary dental insurance plan and the healthcare provider being out-of-network includes the steps of identifying a specific data element from the SPSS as a provider designation with the NCD during Step F, if the matched primary insurance plan is a primary dental insurance plan. This helps ensure that the corresponding healthcare provider is out-of-network within the primary dental insurance plan. If the provider designation is identified as an out-of-network designation, a primary write-off value is zeroed with the NCD. In addition, the primary dental insurance values are calculated based on the zeroed primary write-off value and the SPSS with the NCD. In the preferred embodiment, the primary dental insurance values includes, but are not limited to, the primary insurance payment and the patient responsibility. Finally, the summary report is amended with the primary dental insurance values during Step G, if the primary dental insurance values are calculated. The primary dental insurance values are also input into the HPS. Further, the primary dental insurance values are submitted to the corresponding secondary dental insurance plan. The patient-specific statement along with other relevant claim files are attached to the secondary claim to be submitted together.

When the system of the present invention determines that the patient-specific statement corresponds to a secondary insurance plan (i.e., the patient-specific statement is a secondary EOB statement), the system of the present invention further determines whether the corresponding patient insurance plan falls into one of three categories: (i) primary medical insurance plan with a secondary dental insurance plan, (ii) primary dental insurance plan with an in-network secondary dental insurance plan, or (iii) primary dental insurance with an out-of-network secondary dental insurance plan. In addition, the system must check if there is a corresponding primary EOB statement for the current secondary EOB statement. In general, the corresponding primary EOB statement must be processed first, so the current secondary EOB statement must be put on hold until the corresponding primary EOB statement is processed. Thus, the patient insurance records can further include a plurality of primary EOB statements corresponding to a patient's primary EOB statements previously saved on the NCD. The primary EOB statements can be primary medical/dental statements that were previously saved on the NCD.

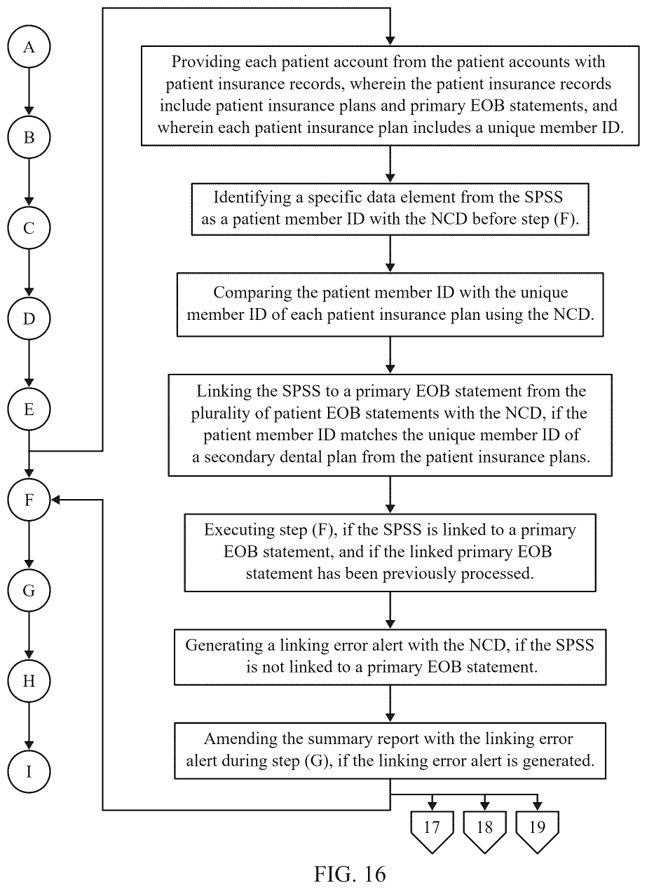

As can be seen in FIG. 16, the subprocess of determining if the patient-specific statement corresponds to a secondary EOB statement and determining if a corresponding primary EOB statement has been processed includes the steps of identifying a specific data element from the SPSS as a patient member ID with the NCD before Step F. Like before, the patient member ID helps accurately determine which patient insurance plan applies to the patient-specific statement. Further, the patient member ID is compared with the unique member ID of each patient insurance plan using the NCD. By comparing the information on the SPSS with the patient records provided by the HPS, the SPSS can be accurately matched to a specific patient insurance plan with the NCD. If the patient member ID matches the unique member ID of a secondary dental plan from the patient insurance plans, the SPSS is also linked to a primary EOB statement from the plurality of patient EOB statements with the NCD.

As can be seen in FIG. 16, after the system links the current secondary EOB statement to a primary EOB statement, Step F is executed, if the SPSS is linked to a primary EOB statement, and if the linked primary EOB statement has been previously processed. As previously discussed, the primary EOB statement needs to be processed first, so if the primary EOB statement has not been processed yet, the current secondary EOB statement is put on hold until the primary EOB statement is processed. Further, a linking error alert is generated with the NCD, if the SPSS is not linked to a primary EOB statement. This way, the corresponding user can be alerted to take the necessary actions, such as manually looking for the corresponding primary EOB statement, or manually processing the primary EOB statement. Further, the summary report is amended with the linking error alert during Step G, if the linking error alert is generated.

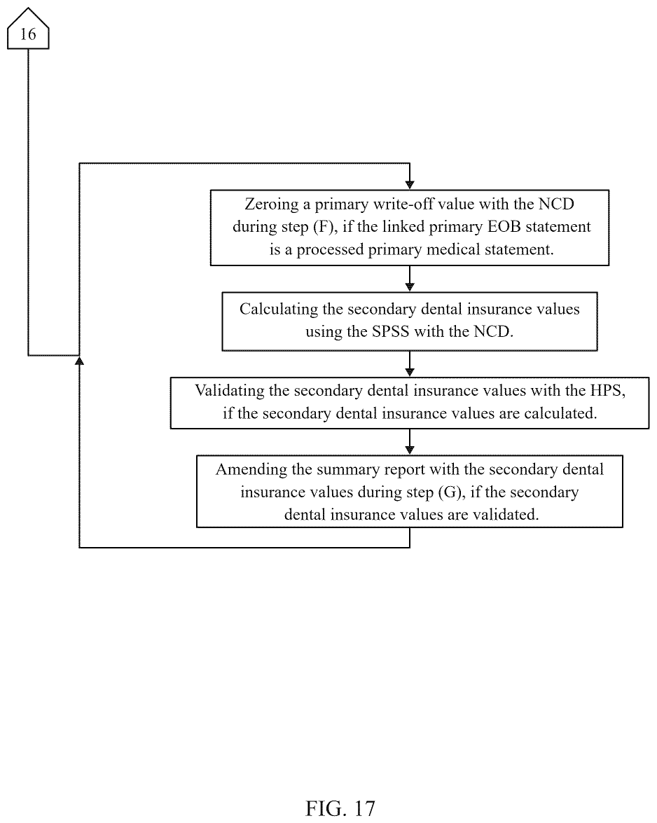

After confirming that the patient-specific statement is a secondary EOB statement and that the linked primary EOB statement has been processed, the system of the present invention can continue to perform the necessary COB procedures. As can be seen in FIG. 17, the subprocess of performing COB for a patient-specific statement with a corresponding primary medical insurance plan and a secondary dental insurance plan includes the steps of zeroing a primary write-off value with the NCD during Step F. This is performed if the linked primary EOB statement is a processed primary medical statement. Once the primary write-off value is zeroed, the secondary dental insurance values are calculated using the SPSS with the NCD. Then, the secondary dental insurance values are verified with the HPS, if the secondary dental insurance values are calculated. Once the validation is performed, the summary report with the secondary dental insurance values during Step G, if the secondary dental insurance values are validated. The validated secondary dental insurance values are also input into the HPS. Alternatively, the corresponding user can be prompted to manually input the secondary dental insurance values.

In a primary medical statement, the system initially analyzes procedure codes included in a secondary dental statement to determine whether the claim relates to preventive or diagnostic procedures. When the procedure codes fail to correspond to preventive codes or diagnostic codes, the system ignores the procedure codes and applies primary insurance payment value into the system. Secondary dental insurance values may include, but are not limited to, a secondary insurance payment, a secondary patient responsibility, and a secondary PPO fee, which are provided as inputs to the HPS.

Further, the secondary dental insurance values are verified using a proprietary process. These proprietary calculations are utilized to perform a validation check and balance to confirm the correctness of the secondary insurance values. First, the secondary insurance payment is calculated using the following formula:

$$CSIP = Secondary PPO fee - PIP$$

Wherein PIP corresponds to primary insurance payment, and CSIP corresponds to calculated secondary insurance payment. If the calculated secondary insurance payment is a positive number, further checks are performed:

$$if ( PIP + SIP \geq Secondary PPO fee )$$

$$CSPR = PIP$$

$$if ( PIP + SIP < Secondary PPO fee )$$

$$CSPR = ( Secondary PPO fee - SIP - PIP ) + PIP = Secondary PPO fee - SIP$$

Wherein PIP corresponds to primary insurance payment, SIP corresponds to secondary insurance payment, and CSPR corresponds to calculated secondary patient responsibility. Alternatively, if the calculated secondary insurance payment is less than or equal to zero, the system redefines the calculated secondary insurance payment as zero. Additional validation is performed to validate the calculated secondary insurance payment, and the secondary payment responsibility matches the values provided by the HPS. If the values are validated, the secondary insurance payment and the secondary payment responsibility are input into the HPS. If not, the corresponding user is alerted and prompted to take over the calculations. In other embodiments, the calculation and the validation of the secondary dental insurance values can be performed differently.

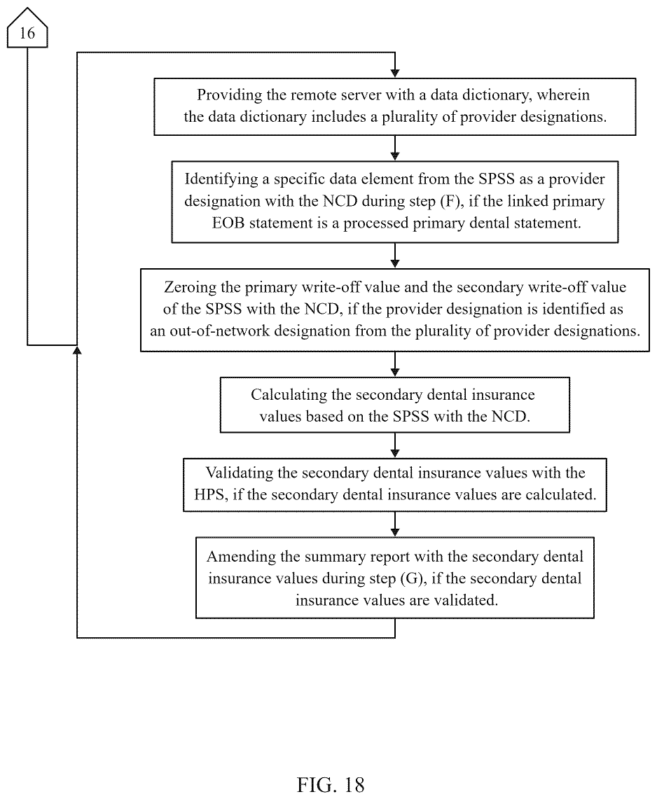

If the SPSS is designated as a secondary dental statement, the system of the present invention must also check if the secondary dental statement is linked to a primary dental statement. In addition, the system must check the provider designation to determine if the corresponding healthcare provider falls in-network or out-of-network. As can be seen in FIG. 18, the subprocess of performing COB for a patient-specific statement with a corresponding primary dental insurance plan and an out-of-network secondary dental insurance plan includes the steps of identifying a specific data element from the SPSS as a provider designation with the NCD during Step F, if the linked primary EOB statement is a processed primary dental statement. This ensures that the current secondary EOB statement corresponds to a claim for an out-of-network healthcare provider.

As can be seen in FIG. 18, if the provider designation is identified as an out-of-network designation from the plurality of provider designations, the primary write-off value and the secondary write-off value are zeroed with the NCD. With the primary and the secondary write-off values zeroed, the secondary dental insurance values are calculated based on the SPSS with the NCD. Further, if the secondary dental insurance values are calculated, the secondary dental insurance values are validated with the HPS. Moreover, if the secondary dental insurance values are validated, the summary report is amended with the secondary dental insurance values during Step G. The verified secondary dental insurance values are also input into the HPS. Alternatively, a dental value error alert is generated with the NCD, if the secondary dental insurance values are not validated. The dental value error alert is output with the NCD to notify the corresponding user so that the secondary dental insurance values can be manually input. Further, the summary report is amended with the dental value error alert during Step G, if a dental value error alert is generated.

With the primary and secondary write-off values zeroed, the secondary dental insurance values calculated and verified preferably correspond to the secondary insurance payment as well as the secondary patient responsibility. These secondary dental insurance values are calculated using the following formulas:

$$CSPR = OF - SIP$$

Wherein CSPR corresponds to calculated secondary patient responsibility, SIP corresponds to secondary insurance payment, and OF corresponds to office fee. The secondary insurance payment and calculated secondary patient responsibility are compared to the values provided by the secondary insurance claim values with the HPS. If confirmed, the secondary insurance payment and secondary patient responsibility are input into the HPS. Alternatively, the corresponding user is alerted and prompted to manually enter the values with the NCD. In other embodiments, different calculations and verification procedures can be implemented.

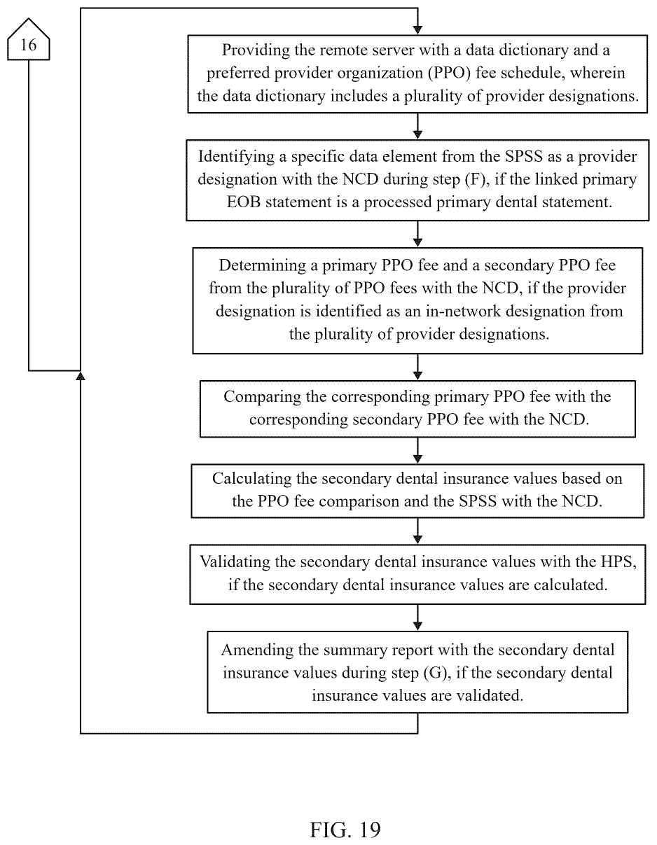

When the secondary EOB statement is determined to have an in-network designation, the system must compare the primary and secondary PPO fee schedule. To do so, the remote server is provided with a PPO fee schedule that includes a plurality of primary PPO fees and a plurality of secondary PPO fees. The PPO fee schedule can also be accessed directly from the HPS via an API connection or other secure protocol. As can be seen in FIG. 19, the subprocess of performing COB for a patient-specific statement with a corresponding primary dental insurance plan and an in-network secondary dental insurance plan includes the steps of identifying a specific data element from the SPSS as a provider designation with the NCD during Step F, if the linked primary EOB statement is a processed primary dental statement. This allows the system to ensure that the current secondary EOB statement corresponds to a healthcare provider that is in-network for the secondary dental insurance plan. Then, a primary PPO fee and a secondary PPO fee from the plurality of PPO fees are determined with the NCD, if the provider designation is identified as an in-network designation from the plurality of provider designations.

As can be seen in FIG. 19, once the corresponding primary and secondary PPO fees are determined, the corresponding primary PPO fee is compared with the corresponding secondary PPO fee with the NCD. Depending on the comparison results, the secondary dental insurance values are calculated based on the PPO fee comparison and the SPSS with the NCD. Different calculation procedures are performed based on the comparison results. Then, the secondary dental insurance values are validated with the HPS, if the secondary dental insurance values are calculated. Like previous EOB procedures, the summary report is amended with the secondary dental insurance values during Step G, if the secondary dental insurance values are validated. The secondary dental insurance values are also input into the HPS if validated. Alternatively, a dental value error alert is generated with the NCD, if the secondary dental insurance values are not validated. Like before, the dental value error alert is output to notify the corresponding user so that the secondary dental insurance values can be manually input. Furthermore, the summary report is amended with the dental value error alert during Step G, if a dental value error alert is generated.

As previously mentioned, different calculation procedures are performed based on the comparison results. When comparing the primary PPO fee with the secondary PPO fee, other factors can be considered such as the corresponding deductible and the annual maximum limit of the corresponding procedures. Based on the comparison results, if the primary PPO fee is greater than the secondary PPO fee, the following calculations are performed:

$$CSIP = Secondary PPO fee - PIP$$

$$if ( PIP \geq Secondary PPO fee )$$

$$CSIP = 0$$

$$CSPR = ( Secondary PPO fee - PIP - SIP ) + PIP = Secondary PPO fee - SIP$$

Wherein PIP corresponds to primary insurance payment, CSIP corresponds to calculated secondary insurance payment, and CSPR corresponds to calculated secondary patient responsibility. If the primary PPO fee is less than the secondary PPO fee, the following calculations are performed:

$$CSIP = Primary PPO fee - PIP$$

$$if ( Lowest PPO fee )$$

$$CSPR = 0$$

$$if ( Highest PPO fee )$$

$$CSPR = ( Secondary PPO fee - PIP - SIP ) + PIP = Secondary PPO fee - SIP$$

Wherein PIP corresponds to primary insurance payment, CSIP corresponds to calculated secondary insurance payment, and CSPR corresponds to calculated secondary patient responsibility. Further, if the primary PPO fee is equal to the secondary PPO fee, the following calculations are performed:

$$CSIP = Primary PPO fee - PIP$$

Finally, all the calculated secondary dental insurance values are validated. In the preferred embodiment, the validated secondary dental insurance values include the secondary insurance payment and the secondary patient responsibility. The calculated secondary insurance payment and the secondary patient responsibility are compared with the values provided by the secondary insurance claim values and the HPS to ensure accuracy. If verified, the secondary insurance payment and the secondary patient responsibility are input into the HPS. If not verified, the user is notified by the output dental value error alert and prompted to manually input the values. In other embodiments, different calculations and validation procedures can be implemented.

As previously discussed, the summarization of the EOB processing is a unique feature of the present invention. With automatization of billing processes, the end user is often left without knowing total input that was done if multiple EOB statements are processed. The summary report shows the calculated insurance values of the primary and secondary EOB statements, the insurance values input into the HPS, the secondary insurance claims being sent out, and the cumulative amount of EOB payments. Further, the summary report includes all the error alerts that were generated during the EOB processing. In the preferred embodiment, these error alerts also have a link to the patient account on the HPS via API so that the end user can follow up with the corresponding carrier on the reasons for the errors with the guidance of the normalized explanation description.

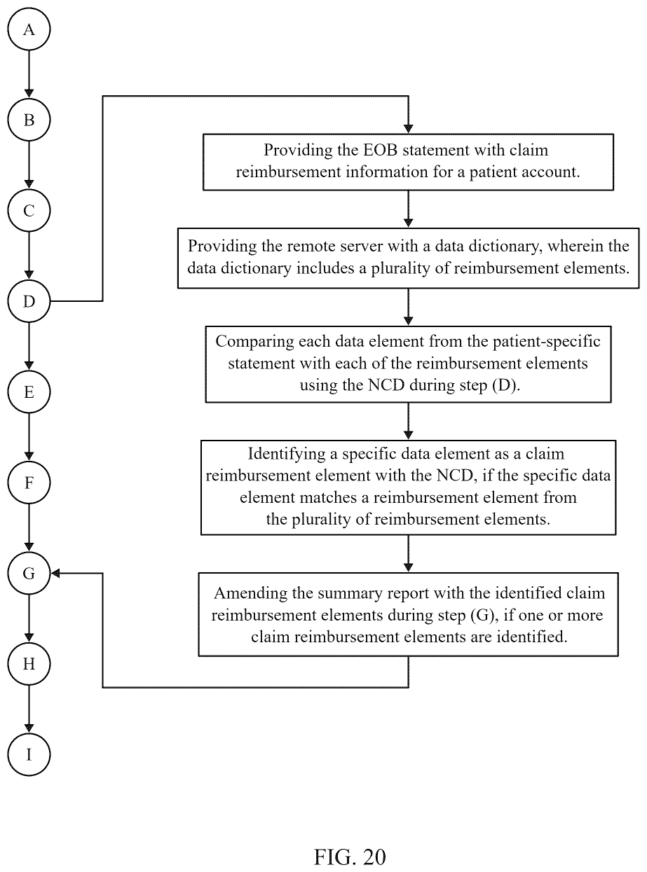

Further, the summary report preferably includes the check/EFT values of the processed EOB statements summarized by the corresponding insurance carrier. These values can include the check number, the check total amount, the patient name, date of service, and the claim processed payment amount. To do so, the EOB statement includes claim reimbursement information for a patient account that the system can access to include the corresponding values into the summary report. In addition, the data dictionary includes a plurality of reimbursement elements that helps the system identify the appropriate reimbursement values. As can be seen in FIG. 20, the subprocess of including reimbursement values in the summary report includes the steps of comparing each data element from the patient-specific statement with each of the reimbursement elements using the NCD during Step E. The system needs to locate the reimbursement elements to include the appropriate values in the summary report.

Further, a specific data element is identified as a claim reimbursement element with the NCD, if the specific data element matches a reimbursement element from the plurality of reimbursement elements, as can be seen in FIG. 20. The claim reimbursement element can be the check number, the check total amount, the patient name, date of service, etc. Further, the summary report is amended with the identified claim reimbursement elements during Step G, if one or more claim reimbursement elements are identified. All identified claim reimbursement elements are included for future auditing. Thus, the summary report allows authorized users such as the owner or billing administrator to follow up on daily transactions. In other embodiments, the summary report can include other information from the processed EOB statement.

Although the invention has been explained in relation to its preferred embodiment, it is to be understood that many other possible modifications and variations can be made without departing from the spirit and scope of the invention.

What is claimed is:

1. A method for processing an explanation of benefits statement from an insurance carrier to a healthcare provider, the method comprising the steps of:
(A) providing at least one network computing device (NCD) managed by a remote server, wherein the remote server includes a plurality of patient accounts, and wherein the NCD is connected to at least one healthcare provider server (HPS);
(B) providing at least one explanation of benefits (EOB) statement, wherein the EOB statement includes dental billing information for at least one patient account;
(C) slicing the EOB statement into at least one patient-specific statement with the NCD;
(D) extracting data elements from the patient-specific statement using optical character recognition (OCR) with the NCD to generate a machine-readable statement;
(E) normalizing the patient-specific statement into a standardized patient-specific statement (SPSS) with the NCD;
(F) performing coordination of benefits (COB) based on the SPSS with the NCD, if the patient-specific statement is normalized;
(G) generating a summary report based on the COB results and the SPSS with the NCD, if the COB is performed;
(H) relaying the patient-specific statement, the SPSS, and the summary report from the NCD to the remote server and to the corresponding HPS; and
(I) inserting the patient-specific statement, the SPSS, and the summary report into the corresponding patient account from the plurality of patient accounts with the remote server.

2. The method as claimed in claim 1 further comprising the steps of:
providing the EOB statement as a physical EOB statement;
capturing the physical EOB statement with the NCD before step (C) to generate a digital EOB statement;
importing the digital EOB statement with the NCD; and
executing step (C), if the digital EOB statement is imported.

3. The method as claimed in claim 1 further comprising the steps of:
providing the EOB statement as a digital EOB statement stored on at least one insurance carrier server;
relaying the digital EOB statement from the insurance carrier server to the NCD before step (C);
importing the digital EOB statement with the NCD; and
executing step (C), if the digital EOB statement is imported.

4. The method as claimed in claim 1 further comprising the steps of:
providing the EOB statement with dental billing information for a plurality of patient accounts;
slicing the EOB statement into a plurality of patient-specific statements with the NCD during step (C); and
performing a plurality of iterations of steps (D) through (I) for each of the patient-specific statements.

5. The method as claimed in claim 1 further comprising the steps of:
providing the remote server with a data dictionary, wherein the data dictionary includes a plurality of patient identifiers;
comparing each data element from the patient-specific statement with each of the patient identifiers using the NCD before step (D);
identifying a specific data element as a patient identifier element with the NCD, if the specific data element matches a patient identifier from the plurality of patient identifiers;
tokenizing each patient identifier element with a unique identifier using the NCD; and
updating the patient-specific statement with the unique identifiers using the NCD.

6. The method as claimed in claim 1 further comprising the steps of:
providing the remote server with a data dictionary, wherein the data dictionary includes a plurality of claim data points and a plurality of standardized data points, wherein each of the claim data points is associated with a corresponding standardized data point;
comparing each data element from the patient-specific statement with each of the claim data points using the NCD during step (E);
identifying a specific data element as a claim data element with the NCD, if the specific data element matches a claim data point from the plurality of claim data points;
overwriting each claim data element with the corresponding standardized data point using the NCD; and
updating the patient-specific statement with the standardized data points using the NCD to convert the patient-specific statement into a SPSS.

7. The method as claimed in claim 6 further comprising the steps of:
validating each standardized data point with the HPS;
executing step (F), if each standardized data point is validated;
generating a claim point error alert with the NCD, if one or more standardized data points are not validated; and
amending the summary report with the claim point error alert during step (G) using the NCD, if a claim point error alert is generated.

8. The method as claimed in claim 1 further comprising the steps of:
providing each patient account from the plurality of patient accounts with a patient insurance record, wherein the patient insurance record includes a primary insurance plan, and wherein the primary insurance plan includes a unique member identification (ID);
identifying a specific data element from the SPSS as a patient member ID with the NCD before step (F);
comparing the patient member ID with the unique member ID using the NCD;
matching the SPSS to the primary insurance plan with the NCD, if the patient member ID matches the unique member ID of the primary insurance plan; and
executing step (F), if the SPSS is matched to the primary insurance plan.

9. The method as claimed in claim 8 further comprising the steps of:
calculating primary insurance values based on the SPSS with the NCD during step (F), if the SPSS is matched to the primary insurance plan;
validating the primary insurance values with the HPS, if primary insurance values are calculated;
amending the summary report with the primary insurance values during step (G), if the primary insurance values are validated;
generating a value error alert with the NCD, if the primary insurance values are not validated; and
amending the summary report with the value alert during step (G) using the NCD, if a value error alert is generated.

10. The method as claimed in claim 1 further comprising the steps of:
providing each patient account from the plurality of patient accounts with patient insurance records, wherein the patient insurance records include a plurality of patient insurance plans, and wherein each patient insurance plan includes a unique member ID;
identifying a specific data element from the SPSS as a patient member ID with the NCD before step (F);
comparing the patient member ID with the unique member ID of each patient insurance plan using the NCD;
matching the SPSS to a specific patient insurance plan with the NCD, if the patient member ID matches the unique member ID of the specific patient insurance plan; and
executing step (F), if the SPSS is matched to a primary insurance plan from the patient insurance plans.

11. The method as claimed in claim 10 further comprising the steps of:
providing the remote server with a data dictionary, wherein the data dictionary includes a plurality of valid procedure codes;
identifying specific data elements from the SPSS as procedure codes with the NCD during step (F), if the matched primary insurance plan is a primary medical insurance plan;
determining the code value of an identified procedure code with the NCD, if the identified procedure code matches a valid procedure code from the plurality of valid procedure codes;
calculating primary medical insurance values based on the determined code values with the NCD, if one or more code values are determined
validating the primary medical insurance values with the HPS, if primary medical insurance values are calculated; and
amending the summary report with the primary medical insurance values during step (G), if the primary medical insurance values are validated.

12. The method as claimed in claim 11, wherein the plurality of valid procedure codes include a plurality of preventive codes and a plurality of diagnostic codes.

13. The method as claimed in claim 10 further comprising the steps of:
providing the remote server with a data dictionary, wherein the data dictionary includes a plurality of provider designations, and wherein the provider designations includes an in-network designation;
identifying a specific data element from the SPSS as a provider designation with the NCD during step (F), if the matched primary insurance plan is a primary dental insurance plan;
calculating the primary dental insurance values based on the SPSS with the NCD, if the provider designation is identified as an in-network designation;
validating the primary dental insurance values with the HPS, if the primary dental insurance values are calculated; and
amending the summary report with the primary dental insurance values during step (G), if the primary dental insurance values are validated.

14. The method as claimed in claim 10 further comprising the steps of:
providing the remote server with a data dictionary, wherein the data dictionary includes a plurality of provider designations, and wherein the provider designations includes an out-of-network designation;
identifying a specific data element from the SPSS as a provider designation with the NCD during step (F), if the matched primary insurance plan is a primary dental insurance plan;
zeroing a primary write-off value with the NCD, if the provider designation is identified as an out-of-network designation;
calculating the primary dental insurance values based on the zeroed write-off value and the SPSS with the NCD; and
amending the summary report with the primary dental insurance values during step (G), if the primary dental insurance values are calculated.

15. The method as claimed in claim 1 further comprising the steps of:
providing each patient account from the plurality of patient accounts with patient insurance records, wherein the patient insurance records includes a plurality of patient insurance plans and a plurality of primary EOB statements, and wherein each patient insurance plan includes a unique member ID;
identifying a specific data element from the SPSS as a patient member ID with the NCD before step (F);
comparing the patient member ID with the unique member ID of each patient insurance plan using the NCD;
linking the SPSS to a primary EOB statement from the plurality of patient EOB statements with the NCD, if the patient member ID matches the unique member ID of a secondary dental plan from the patient insurance plans;
executing step (F), if the SPSS is linked to a primary EOB statement, and if the linked primary EOB statement has been previously processed;
generating a linking error alert with the NCD, if the SPSS is not linked to a primary EOB statement; and
amending the summary report with the linking error alert during step (G), if the linking error alert is generated.

16. The method as claimed in claim 15 further comprising the steps of:
zeroing a primary write-off value with the NCD during step (F), if the linked primary EOB statement is a processed primary medical statement;
calculating the secondary dental insurance values using the SPSS with the NCD;
validating the secondary dental insurance values with the HPS, if the secondary dental insurance values are calculated; and
amending the summary report with the secondary dental insurance values during step (G), if the secondary dental insurance values are validated.

17. The method as claimed in claim 15 further comprising the steps of:
providing the remote server with a data dictionary, wherein the data dictionary includes a plurality of provider designations;
identifying a specific data element from the SPSS as a provider designation with the NCD during step (F), if the linked primary EOB statement is a processed primary dental statement;
zeroing the primary write-off value and the secondary write-off value of the SPSS with the NCD, if the provider designation is identified as an out-of-network designation from the plurality of provider designations;
calculating the secondary dental insurance values based on the SPSS with the NCD;
validating the secondary dental insurance values with the HPS, if the secondary dental insurance values are calculated; and
amending the summary report with the secondary dental insurance values during step (G), if the secondary dental insurance values are validated.

18. The method as claimed in claim 15 further comprising the steps of:
providing the remote server with a data dictionary and a preferred provider organization (PPO) fee schedule, wherein the data dictionary includes a plurality of provider designations;
identifying a specific data element from the SPSS as a provider designation with the NCD during step (F), if the linked primary EOB statement is a processed primary dental statement;
determining a primary PPO fee and a secondary PPO fee from the plurality of PPO fees with the NCD, if the provider designation is identified as an in-network designation from the plurality of provider designations;
comparing the corresponding primary PPO fee with the corresponding secondary PPO fee with the NCD;
calculating the secondary dental insurance values based on the PPO fee comparison and the SPSS with the NCD;
validating the secondary dental insurance values with the HPS, if the secondary dental insurance values are calculated; and
amending the summary report with the secondary dental insurance values during step (G), if the secondary dental insurance values are validated.

19. The method as claimed in claim 1 further comprising the steps of:
providing the EOB statement with claim reimbursement information for a patient account;
providing the remote server with a data dictionary, wherein the data dictionary includes a plurality of reimbursement elements;
comparing each data element from the patient-specific statement with each of the reimbursement elements using the NCD during step (D);
identifying a specific data element as a claim reimbursement element with the NCD, if the specific data element matches a reimbursement element from the plurality of reimbursement elements; and
amending the summary report with the identified claim reimbursement elements during step (G), if one or more claim reimbursement elements are identified.
